(19) United States
(12) Patent Application Publication
(10) Pub. No.: US 2005/0137711 A1
(43) Pub. Date: Jun. 23, 2005

(54) Pre-stressed implant

(76) Inventors: Carleton B. Southworth, Warsaw, IN (US); Paul Tomaszewski, Columbia City, IN (US)

Correspondence Address: MAGINOT, MOORE & BECK;BANK ONE CENTER/TOWER, 1111 MONUMENT CIRCLE, INDIANAPOLIS, IN, 46204, US

(21) Appl. No.: 10/742,294

(22) Filed: Dec. 19, 2003

(51) Int. Cl. A61F 2/36; A61F 2/30

(52) U.S. Cl. 623/22.46; 623/23.46; 623/23.18

(57) ABSTRACT

A pre-stressed orthopaedic implant includes a tension member extending through a bore in the implant. The tension member can include a bolt that is tightened into threads at the closed end of the bore, thereby compressing the implant between the bolt head and the threaded engagement. In another embodiment, the tension member is a spring or a tension cable.

### BACKGROUND OF THE INVENTION

The present invention relates to orthopaedic implants or prostheses, and particularly to implants subjected to high tensile loads. The invention has particular application to implants or prosthesis that form part of a joint of the human body, such as the hip, knee or shoulder.

Implants or joint prostheses have improved significantly over the last few decades, largely due to improvements in the bio-compatibility, strength and durability of the implant materials. New machining processes and material coatings have been developed that enhance the fixation of the implant within the natural bone of a patient. Alloys and ceramics have been developed that emulate the strength of natural bone, while still preserving the biomechanical attributes of the joint being repaired.

In a typical implant or prosthesis, a stem is inserted into the medullary canal of a long bone, such as the femur or humerus. Bone cement or a bone ingrowth coating can be introduced to fix the implant within the bone. The proximal end of the implant can be configured to replace the damaged portion of the patient's natural bone or joint. For instance, in a hip implant, the head of the femur can be removed and an implant utilized that fills the space left by the removed bone. The implant can include a ball to mate with the articulating socket of the hip joint.

The head of the femur is the strongest bone of the human body. It endures significant loads through millions of cycles and a variety of movements during the normal lifespan. Any implant used in the hip must be capable of enduring the same loads without fracture. Strength and durability issues become more acute with smaller implants. Stress concentrations in the region between the stem and head portions of smaller implants can become problematic. While stronger materials have been developed to extend the life of all implants, including hip prostheses, there is always a desire to improve the strength and durability of the implants even further.

Smaller implants are particularly susceptible to mid-stem fractures. The smaller implants are necessary to meet the anatomic constraints of smaller patients. Consequently, there is no room to increase the cross-section of the implants to add strength. The need for increasing resistance to flexure loads is particularly critical with these smaller implants.

### SUMMARY OF THE INVENTION

In order to meet the need for stronger and more durable implants, the present invention contemplates a pre-stressed implant that is especially suited to endure high cyclic tensile loading. In one aspect of the invention, a tension member is fixed within a bore of the implant. This tension member places the implant in compression. When the implant is subjected to loads, the resulting tensile forces acting on the implant act, at least initially, to reduce the compressive load that is generated on the implant by the tension member. In other words, the applied tensile forces de-compress the implant before the implant experiences any meaningful tensile loads. The implant can readily withstand the compressive loads exerted on it by the tension member without any significant risk of failure or fatigue. Moreover, pre-stressing the implant opens up the universe of acceptable materials for the construction of the implant. For instance, the implant can be formed of a high strength ceramic in lieu of the typical metal alloy.

In a preferred embodiment of the invention, the tension member comprises a bolt that extends through a bore in the implant. Where the implant is a hip prosthesis, the bore can extend through the stem or neck/head of the prosthesis. The bore is preferably open at one end of the prosthesis and terminates in internal threads at a closed end of the bore. The head of the bolt bears against the prosthesis at the open end of the bore so that the bolt is put in tension as it is tightened into the internal threads. The bolt tension compresses the prosthesis along the axis of the bore. It is contemplated that the bolt will be threaded into the prosthesis prior to implantation within the bone. The bolt can be tightened to a pre-determined torque that corresponds to an appropriate amount of tension in the bolt, and consequently compression in the implant.

In one specific embodiment, the open end of the bore is disposed at the proximal face of the implant. In another embodiment, the bore opens at the distal end of the implant—i.e., the part of the implant that is buried within the bone. With this embodiment, the head of the bolt can be covered at the distal end of the implant, such as by an end cap. In an alternative configuration, the end cap itself serves as the head of the bolt. The end cap can be configured to be independently coupled to the remainder of the implant, although the bolt tension may be sufficient to hold the end cap in position throughout the life of the prosthesis.

The bolt is sized relative to the bore in the implant to leave a pre-determined gap between the bolt and the inner wall of the implant bore. This gap is calibrated to allow a certain amount of flexure in the implant without causing a commensurate flexure in the tension member.

In a further aspect of the invention, the tension member can be a tension cable. The cable can be a wound multi-filament, pre-stretched cable. The ends of the cable are engaged to caps that are configured to mate with the prosthesis at opposite ends of the bore through the prosthesis. In one embodiment, the bore extends through the entire dimension of the implant so that it is open at its opposite ends. One cap is configured to engage one open end of the bore and to provide an anchorage for the cable. The other cap is configured to engage the opposite end of the bore and to permit tightening of the cable when the cable extends through the bore.

It is an important object of the invention to improve the strength and fatigue resistance of an orthopaedic implant or prosthesis. One benefit achieved by the present invention is that it reduces the tensile stress experienced by the implant. This benefit manifests itself in longer life for the implant.

A further benefit of the invention is that it allows smaller implant dimensions, thereby improving the range of motion for a prosthetic joint. Yet another benefit resides in the ability to use different materials for the implant that might not otherwise be available for traditional designs of the implant. These and other objects and benefits of the invention will become apparent upon consideration of the following written description taken together with the accompanying figures.

### DESCRIPTION OF THE FIGURES

FIG. 1 is side view of an implant according to one embodiment of the invention situated within a long bone of the human body.

FIG. 2 is a cross-sectional view of the implant shown in FIG. 1, taken along line 2-2 as viewed in the direction of the arrows.

FIG. 3 is a side view of an implant according to a further embodiment of the present invention.

FIG. 4 is a partial view of an alternative bolt configuration for use with the implant shown in FIG. 3.

FIG. 5 is a side view of a different configuration for an implant according to the present invention.

FIG. 6 is a partial side view of an alternative configuration for an implant in accordance with the present invention.

FIG. 7 is a side view of an additional embodiment of the invention utilizing a cable as a tension member.

FIG. 8 is a perspective view of one cap for use with the tension cable shown in FIG. 7.

FIG. 9 is a side partial cross-section view of a further embodiment of the invention.

FIG. 10 is a side partial cross-section view of an additional embodiment of the invention.

### DESCRIPTION OF THE PREFERRED EMBODIMENTS

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

In one embodiment of the invention, an implant or prosthesis 10 is disposed within a bone, such as the femur F, as shown in FIG. 1. The implant includes a stem 12 that is engaged within the medullary canal M of the femur. The implant 10 further includes a neck 14 extending from the stem at an appropriate angle dictated by the anatomy of the hip joint. The neck 14 is configured to receive an articulating element, such as a ball joint component B shown in phantom in FIG. 1. The implant 10 can be configured in accordance with known designs as part of a hip prosthesis. It is understood that the elements of the prosthesis 10 can be configured for use at other locations in the body, such as in a shoulder, knee or elbow replacement.

In accordance with one aspect of the present invention, the implant 10 defines a bore 18 passing through the implant. In the embodiment shown in FIG. 1, the bore extends along the longitudinal axis of the stem 12 of the implant. In one embodiment, the bore 18 is open at one end, in this case at the proximal end 12a of the stem, and is closed at the opposite end, or at the distal end 12b as shown in FIG. 1. The bore defines internal threads 20 at the closed end of the bore.

The implant 10 includes a tension member 25 that engages the implant within the bore 18. In the embodiment shown in FIG. 1, the tension member 25 comprises a bolt 27 having a shaft 28 and a head 30. The head 30 is configured to bear against the proximal platform 22 of the implant 10 at the open end of the bore 18. The shaft defines a threaded portion 32, which is situated at the distal end of the bolt in FIG. 1. The threaded portion 32 is configured to engage the internal threads 20 in the bore 18 of the implant 10. The threaded engagement between the threaded portion 32 and the internal threads 20 can be configured to permit calibrated torquing of the bolt 27 to produce a pre-determined tension in the bolt and compression in the implant. The threaded engagement is configured to maintain the bolt in tension throughout the life of the implant, even when subjected to the cyclic tensile loads associated with a typical hip implant.

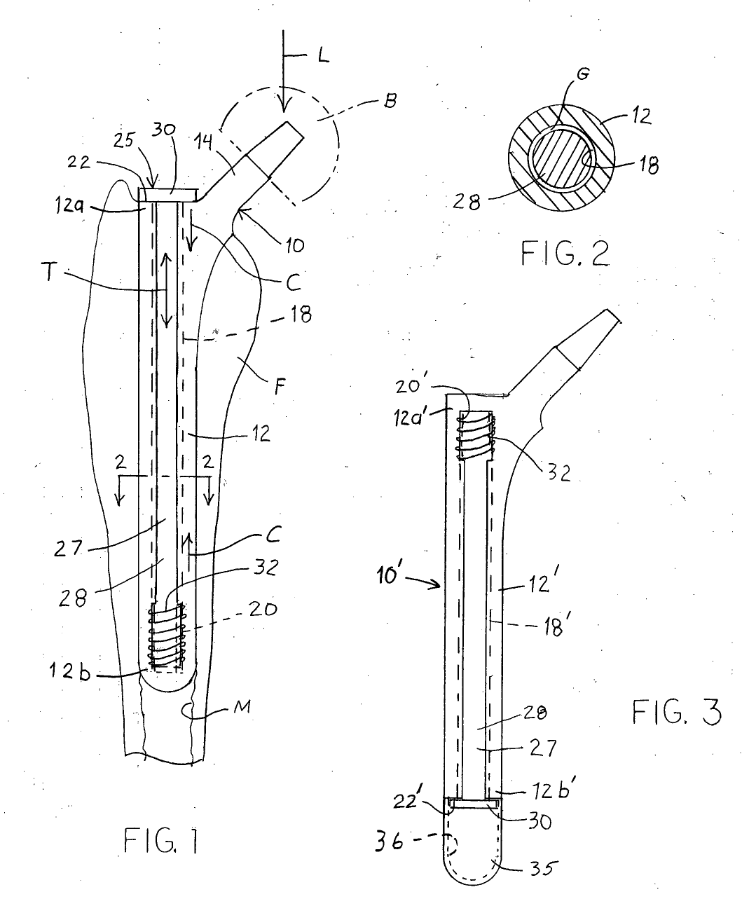

As shown in the cross-sectional view of FIG. 2, the shaft 28 of the bolt 27 has a diameter that is less than the inner diameter of the bore 18 of the stem 12. Preferably, these diameters are calibrated to produce a pre-determined gap G between the bolt and the inner wall of the stem bore. The gap is provided to prevent contact between the stem and the bolt when the implant is loaded. This gap G is sufficient to permit bending of the implant stem 12 during cyclic loading of the implant without also bending the bolt 27. In a specific embodiment, the dimension of the gap G can be about 0.02 inches (0.5 mm).

In certain embodiments, the gap G can also be filled with a material having a high compressive strength. Alternatively, only portions of the gap are filed with an adjunct material. For instance, some parts of the length of the bore 18 can include a series of washers that are formed, for instance, of ceramic, metal or silicone. The material can also be a hardenable material or an incompressible gel.

In the preferred embodiment, the bore 18 extends substantially along the entire length of the stem 12. In addition, the internal threads 20 are situated at the end of the bore. In alternative configurations, the bore can terminate nearer the middle of the implant 10. It is believed that the greatest stress concentration for a hip implant of the type shown in FIG. 1 is at the region below where the neck 14 is integrated into the stem 12. Consequently, the greatest need for pre-stressing is at this region. The proximal half of the implant can be placed under compression with a tension member 25 that extends past this problematic region. Thus, the threaded engagement between the tension bolt 27 and the stem 12 can occur generally at the mid-point of the stem.

In the embodiment of the invention shown in FIG. 1, the tension member 25, or bolt 27, is “top loaded”—i.e., the bolt is threaded in through the proximal end 12a of the implant stem. In an alternative embodiment, shown in FIG. 3, the bolt is loaded from the distal end 12b′ of an implant 12′. Thus, in this embodiment, the bore 18′ is open at the distal end 12b′ and closed at the proximal end 12a′ of the implant. (The bore 18′ can be open at the proximal end 12a′, although less preferable to a closed ended bore). The bore 18′ terminates in threads 20′ to mate with the threaded portion 32 of the bolt. The head 30 of the bolt contacts the distal surface 22′ of the stem to place the stem in compression as the bolt threads 32 are threaded into the internal threads 20′ of the bore.

Since the bolt is introduced from the distal end 12b′ of the implant 10′ of the embodiment in FIG. 3, it is preferable to close the distal end with a cap 35. The cap can include a hollow portion 36 to receive the head 30 of the bolt. The hollow portion 36 can be configured to closely conform to the bolt head. The hollow portion can also be configured to provide a larger cavity, as depicted in FIG. 3, which can be filled with a cement material to fix the bolt head in position and prevent backing out of the bolt from the internal threads 20′. The cement material can also be used to fix the end cap 35 to the distal end 12b′ of the stem 12′. Alternatively, the end cap and stem distal end can include a mating feature, such as a threaded or a snap-fit engagement to hold the end cap on the implant during insertion into the medullary canal M.

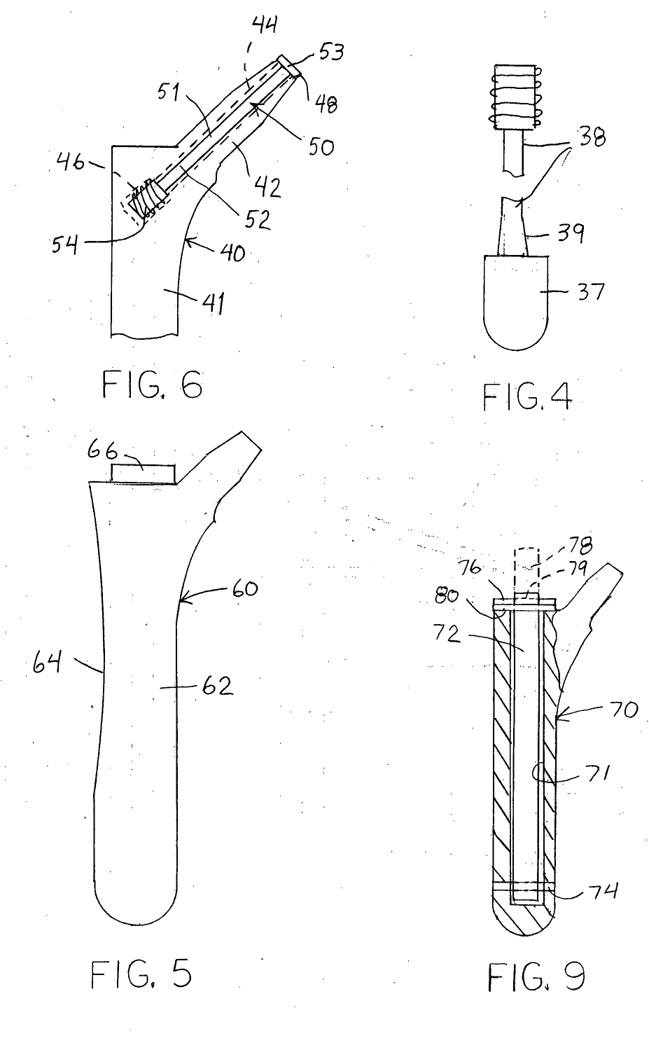

In another embodiment of a distally inserted tension member, a modified end cap 37 can be provided with the tension member 38 attached, as shown in FIG. 4. In this embodiment, the tension member can be integral with the end cap 37. For instance, where the tension member is a bolt, the head of the bolt can be modified to the configuration of the end cap 37. The end cap can be provided with driving flats or other suitable feature for engagement by a driving tool to thread the tension member into the internal threads 20′.

In order to enhance the fixation of the end cap 37 and the tension member 38 to the stem 12′, the tension member can be provided with a tapered portion 39. This tapered portion can mate with a complementary tapered portion at the distal end 12b′ of the bore 18′ (not shown). Preferably, the taper is a self-tightening Morse taper. The provision of a Morse taper interface at the distal end of the tension member 38 can operate as a mechanism to prevent over-tensioning of the element. The orientation of the taper interface is calibrated so that the taper fixation occurs at or after the point at which the tension member is at its pre-determined tension and the implant is at its pre-determined compression.

As depicted in FIG. 4, the end cap 37 is generally limited to the bullet-nose end of the implant. However, the cap 37 with the tension member 38 attached can be of a wide range of lengths. In other words, the cap 37 can form the lower third of the total length of the implant 10′. In this case, the length of the tension member 38 can be reduced accordingly. This alternative embodiment provides a modular implant that can be adjusted to a length appropriate for a particular patient.

In order to improve the overall strength of the implant, an implant 60 can be provided as shown in FIG. 5. The implant includes a stem 62 that receives a tension member 66, which can be of any form discussed herein. The stem 62 can be provided with a curved surface 64 at one side of the implant 60. This curved surface assumes a more circular configuration as the tension member 66 is tightened and the stem 62 is placed in compression. This circular radius can increase the bending resistance of the implant when subjected to the normal cyclic loads L (FIG. 1).

In the previous figures, the tension member has been shown engaged within the portion of the prosthesis that is implanted in the patient's bone. The tension member can also be used at other locations of the prosthesis that are susceptible to bending or tensile loads. For instance, as shown in FIG. 6, an implant 40 includes a stem 41 configured to be fixed within a long bone. The implant also includes a neck 42 on which is mounted an articulating component of a joint, such as the hip joint. The neck 42 defines a bore 44 therethrough that can extend into the proximal part of the stem 41.

The bore 44 is provided with internal threads 46 at its closed distal end and is open at a platform 48 at the proximal end of the neck 42. A tension member 50 is mounted within the bore 44 that is in the form of a bolt 51. The head 53 of the bolt contacts the platform 48 as the threaded portion 54 engages the internal threads 46 of the bore. In this respect, the bore 44 and the tension member 50 can be configured similar to the like components described above. With this embodiment, the neck is placed in compression so that imposition of a load L (FIG. 1) produces tensile forces that first reduce the compression of the neck 42 before the neck endures significant tensile stress.

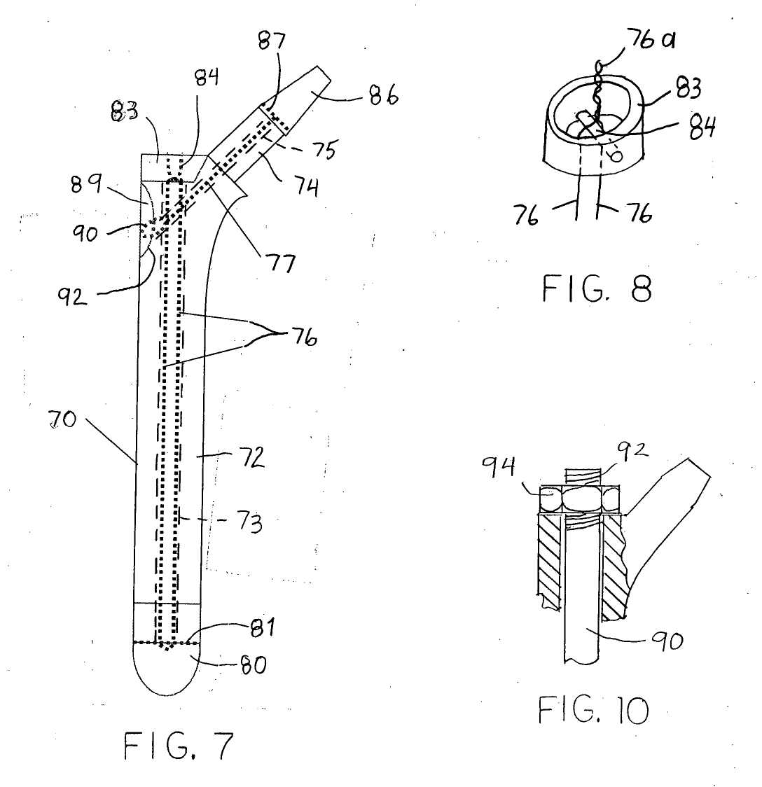

In the embodiments described above, the tension member includes a bolt. The present invention contemplates other elements that are capable of being placed in tension and ultimately capable of compressing at least a portion of an orthopaedic implant or prosthesis. Thus, the tension member can include a cable or a spring system, as depicted in FIG. 7. The implant 70 includes a stem 72 and a neck 74, similar to the stem and neck described above. The stem can include a bore 73 extending throughout the entire length of the stem. Instead of or in addition, the implant 70 can include a bore 75 extending through the neck 74. The bore 75 passes through the neck and through an upper portion of the stem. In this embodiment, both ends of both bores 73 and 75 are open.

The implant 70 can include a tension member 76 passing through bore 73 and a tension member 77 passing through bore 75, if it is present. These tension members can be springs or cables. The opposite ends of these tension members 76, 77 are fastened to caps that close the ends of the bores and anchor the tension members within the implant. For instance, the tension member 76 can be a cable that is fastened to an end cap 80 using an anchor 81 mounted within the cap. The tension cable passes through the bore 73 from the distal end to the proximal end and is fastened to a proximal cap 83 by an anchor 84.

In one embodiment, the tension cable 76 can include a multiple strand pre-stretched cable that is looped around the anchor 81 in the distal cap 80. The two ends of the cable pass through the bore 73 and are twisted around the anchor 84 in the proximal cap 83, as depicted in FIG. 8. The cable can be tensioned and twisted using known cable tensioning devices, such as devices used for cable cerclage procedures. The cable twist 76a can be embedded within the cap 83 and encased in epoxy. Preferably, the two cable segments are twisted as they pass along the length of the bore 73 to enhance their strength in tension.

A similar approach can be followed for the cable 77 passing through the bore 75 in the implant neck 74. In this instance, one cap 86 and anchor 87 are configured to permit attachment of an articulating component, such as the ball B depicted in FIG. 1. The distal end of the cable 77 is fastened to an anchor 90 in a cap 89. The cap 89 fits within a complementary shaped recess 92 defined in the surface of the stem 72. Preferably, the cap 89 is affixed within the recess by mechanical or chemical fastening means. For instance, the cap and recess can form mating Morse tapers.

In certain embodiments of the invention, the tension member can be completely sealed within the implant, allowing the use of non-traditional materials to form the tension member that might be less resistant to the joint environment that more traditional materials. For instance, the tension member can be formed of a “liquid metal”—i.e., a metal created using nanotechnology to have an amorphous structure that resists crack propagation and that provides fatigue strength, yield strength and elastic limit properties superior to traditional crystalline metals. Sealing the tension member within the implant will mitigate potential biocompatibility concerns for such non-traditional materials.

The tension members of the present invention put critical portions of an orthopaedic implant or prosthesis in compression. By so doing, when the critical portion of the implant is exposed to a tensile load, the load must first reduce the implant compression before the critical portion experiences any tensile stress. A proper combination of material compressive strength and tension member tension can significantly reduce the amount of tensile stress experienced at these critical portions of the implant. As a consequence, the implant can be formed of non-traditional materials. These non-traditional materials may be very strong in compression but weaker than the traditional implant metal allows when subject to tensile loads.

Another consequence of the pre-stressed implant of the present invention is that the critical portions of the implant can be constructed with reduced cross-sections. For instance, the neck 74 of the implant 70 shown in FIG. 7 is smaller in cross-section than traditional hip implants. The tension member 77 pre-compresses the neck 74, thereby increasing its bending strength and improving its ability to withstand the normal cyclic loads exerted on the articulating end of the implant.

Other tension members and tension protocols are contemplated by the present invention. For instance, the bolt tension member 25 shown in FIG. 1 can be heated, threaded into the implant and then allowed to cool within the implant. As the tension member cools, it shrinks, thereby placing the bolt in tension. With this approach, the threaded interface between the bolt and the implant body is not required to pre-tension the bolt. Thus, with the heat/cool approach the threaded end 32 of the bolt can be eliminated in favor of some other form of mechanical fixation. For instance, a pin, similar to the pin 84 shown in FIG. 8, can pass transversely through the implant stem and through the end of the tension member 25 to fix the embedded end of the member to the implant body.

In one embodiment, an implant 70, depicted in FIG. 9, includes a tension member 72 in the form of a rod extending through the bore 71 in the implant. A pin 74 passes through aligned bores in both the implant 70 and the rod 72 to fix the distal end of the rod within the bore. The proximal end can also be fixed in place using a similar pin 76 passing through aligned bores in the implant and rod. With this approach, the rod 72 can be pre-heated as described above and then fixed at the distal end using the pin 74. The pin 76 can also put in place to fix the proximal end of the rod. It is contemplated that the opening at the proximal end of the implant for insertion of the pin 74 is situated to correspond to the heated length of the rod.

As an alternative approach, the rod 72 is longer than the implant so that a portion 78 is accessible beyond the end of the implant. This portion 78 can be engaged by a distraction tool operable to pull the proximal end of the rod while the distal end is held fixed with the implant by pin 74. Once the desired tension has been reached, the proximal end can be fastened using the pin 76 or other similar mechanical fastener. With this approach, the opening in the proximal end of the implant 70 for receiving the pin 76 is located to be aligned with the corresponding pin-receiving opening 79 through the rod 72 only when the rod has been appropriately tensioned or stretched. In lieu of passing the pin 76 through an opening at the proximal end of the implant, the pin 76 can bear against a washer 80 that contacts the end of the implant.

As a further alternative to the tension member shown in FIG. 1, the tension member 25 can be threaded at both ends. With this alternative, the head 30 would constitute a nut or lock nut arrangement, such as the nut 94 shown in FIG. 10, which would engage corresponding threads 92 at the proximal end of a tension member 90. The member 90 can be tensioned by tightening the nut 94 down onto the proximal threads 92 until a desired torque is reached. In this case, the proximal threaded portion would extend beyond the end of the implant, with the excess length severed once the implant has been pre-tensioned. With this approach, the distal threaded portion 32 found on the tension member 25 can be eliminated in favor of the pin approach discussed above.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

1. A bone implant comprising:
an elongated stem configured to be disposed within a bone, said stem having a proximal end and a distal end;
a bore defined in said elongated stem, open at least at said proximal end; and
a tension member configured to extend into said bore, and having a proximal portion engaging said stem between said proximal and distal ends and a distal portion engaging said stem between said proximal portion of said tension member and said distal end of said stem,
whereby said tension member is placed in tension between said proximal portion and said distal portion.

2. The bone implant according to claim 1, wherein:
said tension member includes a shaft defining threads at said distal portion and an enlarged head portion associated with said shaft at said proximal portion; and
said elongated stem defines internal threads in said bore for engagement with said threads of said tension member, said internal threads disposed within said bore so that said enlarged head contacts said elongated stem at said proximal end as said threads are threaded into said internal threads.

3. The bone implant according to claim 2, wherein said enlarged head portion of said tension member is integral with said shaft.

4. The bone implant according to claim 2, wherein:
said enlarged head portion of said tension member is a nut; and
said shaft includes threads at said proximal portion of said tension member for mating with said nut.

5. The bone implant according to claim 1, wherein said tension member is a cable having first means at said proximal portion for engaging said elongated stem and second means at said distal portion for engaging said elongated stem to place said cable in tension.

6. The bone implant according to claim 1, wherein said tension member is a tension spring having first means at said proximal portion for engaging said elongated stem and second means at said distal portion for engaging said elongated stem to place said cable in tension.

7. The bone implant according to claim 1, wherein said stem is configured for engagement within a femur.

8. The bone implant according to claim 1, wherein:
said tension member is configured to define a gap between said tension member and said bore when said tension member extends therethrough.

9. The bone implant according to claim 8, wherein said gap is filled with a material having a compressive strength that is relatively greater than the compressive strength of said elongated stem.

10. The bone implant according to claim 1, wherein at least one of said proximal portion and said distal portion of said tension member is connected to said stem by a pin.

11. A femoral implant comprising:
a stem portion configured to be engaged within the femur;
a head portion including an elongated neck having a proximal end and an opposite distal end, said neck connected to said stem portion at said distal end and defining an articulating joint component at the proximal end;
a bore defined from said proximal end to said distal end of said neck, said bore open at said proximal end; and
a tension member configured to extend into said bore, and having a proximal portion configured to engage said neck between said proximal and distal ends and a distal portion configured to engage said bore adjacent said distal end of said neck,
whereby said tension member is placed in tension between said first end and said second end.

12. The femoral implant according to claim 11, wherein:
said bore extends through said neck and into said stem portion; and
said second end of said tension member engages said bore within said stem portion.

13. The femoral implant according to claim 11, wherein said first end of said bore is defined in said articulating joint component.

14. The femoral implant according to claim 13, wherein said bore defines a countersunk bore portion within said articulating joint component.

15. A method for implanting an implant into a bone comprising the steps of:
placing an implant under compression; and
fixing the implant within a bone while the implant is under compression.

16. The method for implanting an implant according to claim 15, wherein the step of placing the implant under compression includes:
engaging a tension member to the implant; and
placing the tension member in tension.

17. The method for implanting an implant according to claim 16, wherein:
the implant includes a bore; and
the step of engaging a tension member to the implant includes engaging the tension member to a proximal and a distal portion of the bore.

18. The method for implanting an implant according to claim 17, wherein:
the tension member is a bolt having an enlarged head at its proximal end and threads at its distal end; and
the step of engaging a tension member to the implant includes threading the distal end of the bolt into corresponding threads at the distal portion of the bore so that the enlarged head engages the implant at the proximal portion of the bore.

19. The method for implanting an implant according to claim 16, wherein the step of placing the tension member in tension includes heating the member prior to engaging the member to the implant and then allowing the member to cool when the member is engaged to the implant.

20. The method for implanting an implant according to claim 15, wherein the step of placing an implant under compression occurs before the step of fixing the implant in a bone.
